(19) United States
(12) Patent Application Publication
(10) Pub. No.: US 2011/0097740 A1
(43) Pub. Date: Apr. 28, 2011

(54) REAL-TIME CONTINUOUS DETECTION DEVICE

(75) Inventors: Se Hwan Paek, Seoul (KR); Hyun-Kyu Cho, Suwon-si (KR)

(73) Assignee: KOREA UNIVERSITY RESEARCH AND BUSINESS FOUNDATION, Seoul (KR)

(21) Appl. No.: 12/999,979

(22) PCT Filed: Jun. 12, 2009
(86) PCT No.: PCT/KR09/03174

§ 371 Date: Dec. 17, 2010

(30) Foreign Application Priority Data
Jun. 18, 2008 (KR) 10-2008-0057532

(51) Int. Cl. G01N 33/543; C12M 1/34; G01N 33/53; B82Y 15/00

(52) U.S. Cl. 435/7.9; 435/287.1; 436/518; 422/69; 977/773; 977/920

(57) ABSTRACT

Provided is a real-time continuous detection device for detecting an analyte in a sample including: a sample inflow channel; a sample assay site; and a sample outflow channel, wherein the sample assay site includes a reversible capturing recognizing component and a sensor which detects a signal generated from a binding body of the analyte and the reversible capturing recognizing component. According to the real-time continuous detection device, it is possible to measure a change in concentration of the analyte in real time by continuously recycling the reversible capturing recognizing component. The real-time continuous detection device can be used to detect or assay living organism metabolites, a protein, a hormone, a nucleic acid, a cell, a food test material, an environment contaminant, national-defense chemical, biological and radiological test materials, or the like. Accordingly, the real-time continuous detection device can be applied to medical, public health, national defense, environment, food, veterinary, and biotechnology industries.

### TECHNICAL FIELD

The present invention relates to a real-time continuous detection device, and more particularly, to a real-time detection device for detecting an analyte including a sample inflow channel, a sample assay site, and a sample outflow channel, wherein the sample assay site includes a reversible capturing recognizing component and a sensor which detects a signal generated from a binding body of an analyte and the capturing recognizing component.

### BACKGROUND ART

In various fields such as health medical, food, environment, veterinary, and national defense fields, assay methods using a specific recognizing reaction such as an antigen-antibody binding reaction and a nucleic acid hybridization reaction has been used for detecting organic materials having complicated structures, particularly, protein, hormone, nucleic acid, cell, or the like. A biological recognizing reaction has high specificity and high affinity. Therefore, various types of assay systems using the biological recognizing reaction and the conventional assay principle have been developed.

As an example of the developed immunoassay system, a solid-phase immunoassay (for example: enzyme-linked immunosorbent immunoassays; ELISA) method using a microtiter plate as a fixation body has been applied to various diagnosis and assay fields (Irina Ionescu-Matiu et al., J Virol Methods, Vol. 6 (1), Page 41-52, 1983; Christopher Heeschen et al., Clinical chemistry, Vol. 45 (10), Page 1789-1796, 1999). Due to a porous membrane-based reagent-free fast diagnosis kit, immunoassay can be performed without limitation to location such as home (R. Chen et al., 1987, Clin. Chem. Vol. 33, Page 1521-1525; M. P. A. Laitinen, 1996, Biosens. Bioelectron., Vol. 11, 1207-1214; S. C. Lou et al., 1993, Clin. Chem., Vol. 39, 619-624; S. H. Paek et al., Methods Vol. 22, Page 53-60, 2000; S. H. Paek et al., BioChip J. Vol. 1, Page 1-16, 2007).

On the other hand, automatic diagnosis apparatuses for accurate diagnosis have been provided to medical institutes such as hospitals or clinical test centers, and bio sensor array chips for determination of nucleic acid sequence and quantitative assay of an expression degree of protein and lap-on-a-chips for performing a sequential continuous process such as sample preparation for automatic micro assay have been actively researched and developed (Kyeong-Sik Shin et al., Analytical Chemica Acta Vol. 573-574, Page 164-171, 2006). As commercialized examples, there are Custom Array (produced by CombiMatrix (USA)) for search of genomics, Verigene ID platform (produced by Nanosphere (USA)) for measurement of single nucleic acid polymorphism (SNP), GeneChip System (produced by AffyMetrix (USA)), BioDetect Test Card (produced by Integrated NanoTechnologies (USA)) for in-situ assay, and the like.

Recently, nano-bio sensor technology, that is, a fusion of nanotechnology and biotechnology has drawn attention as a 21st century advanced technology. Associated original technology has been actively researched worldwide as well as domestically. Several institutes have concentrated on development of ultra-sensitive bio sensor technology. However, the technology is still in the beginning stage, and a nanosensor concept (Yi Cui et al., Science, Vol. 293, Page 1289-1292, 2001; Jong-in Hahm et al., Nanolett., Vol. 4, Page 51-54, 2004), a vibration type cantilever-based immunoassay (Y Arntz et al., Nanotechnology, Vol. 14, Page 86-90, 2003) or the like are reported.

With respect to the recognizing components such as antibodies used for most of existing assay systems, a washing step is necessarily performed in order to separate a binding body after the binding of an analyte and the recognizing component. In this case, in order to minimize a loss of the formed binding body due to the washing step, the recognizing component needs to have a very low dissociation rate. Therefore, once the analyte is bound, the analyte cannot be detached from the recognizing component. Accordingly, most of the sensors cannot be continuously used, and the sensors may be used as only a disposable sensor. Recently, a non-invasive sensing method for monitoring glucose has been very actively researched (Ronald T. Kurnik et al., Sensors and Actuators B: Chemical, Vol. 60, Page 19-26, 1999). Particularly, demands for continuous measurement of marker materials associated with diseases of critically severe patients have been greatly increased in hospitals, but the demands cannot be satisfied due to technical problems. However, in the case the reaction between the analyte and the recognizing component is reversibly operated, various diseases can be continuously monitored in real time.

If the continuous measurement of the analyte is possible, bio sensors which a human can wear or which can be planted in a human body will be developed in the future. If the sensor having the reversible recognizing component is used, biological information is continuously measured and diagnosed, so that common diseases such as infectious disease or adult disease of high risk group patients (chronic patients, old persons, or the like) having relatively high probability of disease occurrence can be early monitored and managed. Therefore, in the U-health care age providing the health care environment where medical service can be made anytime and anywhere on the basis of Ubiquitous computing environment, the real-time assay tools will be essential for preventive medicine in the future (Anthony P F Turner, Nature Biotechnology, Vol. 15, Page 421-421, 1997). If the U-health care environment is implemented, existing medical paradigm concentrated on treatments in the hospital after the disease occurrence will be greatly changed, and chronic patients, old persons, or the like need not be hospitalized for a long time.

As a result of the research and development for solving the problems of the conventional technologies, the inventors found out that, in the case of introducing a reversible capturing recognizing component into a real-time detection device for detecting an analyte and continuously recycling the recognizing component, a signal is generated in real time according to a change in concentration of the analyte participated in the reaction, so that continuous measurement of the analyte can be performed by measuring the signal, and the inventors completed the present invention.

### DISCLOSURE

### Technical Problem

The present invention is to provide a real-time continuous detection device for detecting an analyte which is capable of continuously recycling a reversible capturing recognizing component by introducing the reversible capturing recognizing component.

The present invention is also to provide a real-time continuous detection method for detecting an analyte using the real-time continuous detection device.

The present invention is also to provide a method of selecting a reversible capturing recognizing component used for the real-time continuous detection device.

### Technical Solution

According to an aspect of the present invention, there is provided a real-time continuous detection device for detecting an analyte 11 including: a sample inflow channel; a sample assay site; and a sample outflow channel, wherein the sample assay site includes a reversible capturing recognizing component 10 and a sensor which detects a signal generated from a binding body of the analyte 11 and the reversible capturing recognizing component 10 (refer to (A) of FIG. 1).

In the present invention, the aforementioned analyte denotes a material which is injected into the surface of the sensor so as to be detected by using the sensor included in the sample assay site, the aforementioned capturing recognizing component denotes a material which is fixed on the sensor chip in the sample assay site so as to specifically bind with and capture the analyte in the sample assay site, and the aforementioned binding body denotes a conjugate formed by the binding of the analyte and the reversible capturing recognizing component.

For example, in the case the analyte is an antigen or a ligand, the capturing recognizing component is an antibody corresponding to the antigen or a receptor corresponding to the ligand. On the contrary, in the case where the analyte is an antibody corresponding to an antigen or a receptor corresponding to a ligand, the capturing recognizing component is the antigen or the ligand.

In the present invention, the reversible capturing recognizing component denotes a capturing recognizing component having a reaction kinetics characteristic of high association (attachment) and dissociation (detachment) rates and high affinity. In the case where the capturing recognizing component having high association and dissociation rate constants is used, even though the recognizing component is continuously used, a high sensitivity of assay can be maintained. Herein, the affinity can be represented by an equilibrium association constant. The equilibrium association constant K_{A} is defined by (association rate constant kₐ)/(dissociation rate constant k_{d}). In the present invention, for example, an antibody having a reversible reaction characteristic and a high affinity is used, highly sensitive real-time continuous detection can be implemented.

If a capturing recognizing component having only the reversible reaction characteristic, of which the affinity is 1×10⁶ L/mol or less, is used, the sensitivity of measurement of the detection device is so low as μmol/L level, there is a problem in that the it is difficult to apply the recognizing component to the detection of the analyte of the bio marker which represents most of diseases or symptoms. This is because, as the affinity of the recognizing component is lowered, the concentration range of the detectable analyte is heightened. For example, if a reversible recognizing component (for example, an antibody) of which the association rate constant is lowered or of which the association rate constant is maintained constant and the dissociation rate constant is too heightened is used, the affinity is lowered down to 1×10⁶ L/mol or less. Therefore, as disclosed in the present invention, a particular method of selecting the reversible recognizing component needs to be introduced in order to obtain a reversible recognizing component having a high affinity. For example, after the fixed antigen and the recognizing component is bound with each other and washed with a neutral buffer solution, the recognizing component which has a low remaining activation value with respect to the concentration of the recognizing component is primarily selected (refer to Embodiment 1). Next, after the association rate constant and the dissociation rate constant are measured by using a sensor (for example, a surface plasmon resonance sensor) fixed with the antigen, the recognizing component having a high affinity is secondarily selected (refer to Embodiment 3). Accordingly, the reversible recognizing component having a high affinity can be effectively produced.

Therefore, in order to comply the reversible capturing recognizing component with the object of the present invention, the recognizing component has a fast reaction kinetics characteristic and a high affinity maintained with the equilibrium association constant of 1×10⁷ L/mol or more. Preferably, the high affinity is maintained with the equilibrium association constant ranging from 1×10⁸ L/mol to 1×10¹² L/mol, and more preferably, the high affinity is maintained with the equilibrium association constant ranging from 1×10⁹ L/mol to 1×10¹² L/mol.

In the real-time continuous detection device according to the present invention, it is preferable that the reversible capturing recognizing component has a reversible reaction characteristic so that an association rate constant kₐ is in a range of from 1×10⁵ Lmol⁻¹ sec⁻¹ to 1×10⁸ Lmol⁻¹ sec⁻¹ and a dissociation rate constant k_{d} is in a range of from 1×10⁻³ sec⁻¹ to 1×10⁻¹ sec⁻¹ and a high affinity so that an equilibrium association constant K_{A}=kₐ/k_{d} is 1×10⁸ L/mol or more at the time of reacting with the analyte in the sample.

In the case where the capturing recognizing component having the features is used for the continuous detection device, since both of the association and dissociation rate constants are high, the response time of the detection device is short, so that the analyte can be detected in real time. In addition, since the equilibrium association constant is also high, high sensitivity of measurement can be obtained. However, in the case the constants deviate the above ranges, particularly, in the case where the dissociation rate constant is lower than the disclosed limit, the analyte cannot be easily detached from the capturing recognizing component, so that the response time in the continuous measurement is too long. Otherwise, in order to facilitate the detachment, severe conditions (for example, acidic pH) are inevitably used, so that it is basically impossible to perform real-time detection. In addition, even in the case where the association and dissociation rate constants are in the given range, if the equilibrium association constant is lower than the disclosed limit, as described above, the sensitivity of assay is lowered, so that practical application is extremely limited.

In general, a monoclonal antibody as a typical recognizing component for a specific analyte can be produced by a hybridoma method where an animal is immunized to the analyte (Kohler. G et al., Nature, Vol. 256, Page 495-497, 1975), a gene recombination method (H P Fell et al., PNAS, Vol. 86, Page 8507-8511, 1989), a phage display method (Nicholas A. Watkins et al., Vox Sanguinis, Vol. 78, Page 72-79, 2000) or the like. In the widely used method of selecting an antibody in a solid-phase immunoassay, since a washing process is used in order to remove excessively remaining components after the reaction, it is difficult to select the reversible antibody which is easily detected from the analyte in the washing process. Therefore, for the reversible capturing recognizing component of the present invention, a particular process for selecting the reversible antibody is required. In the present invention, in order to solve the problem, as an example, a selection system is used, where installed with a label-free sensor such as a surface plasmon resonance sensor which can trace the real-time reaction binding at the time of reaction and washing.

After the analyte (antigen) is fixed on the surface of the sensor, the produced antibody diluted with the carrier solution is continuously injected. And then, if the washing is performed with the same carrier solution, the density of the binding body which is formed or dissociated through the association and dissociation reactions between the antigen and the antibody on the surface is measured from the sensor in real time.

As a detailed example of the present invention, the surface plasmon resonance sensor-based selection system is used in order to select the reversible capturing recognizing component. If a predetermined concentration of the antibody solution appropriately diluted is injected into the system, the signal is increased by the binding reaction as the time elapses. In other words, at the time of washing when the concentration of the antibody is 0 (zero), the density of the binding body is changed according to the reversible reaction characteristic of the antibody (refer to FIG. 2). In most of existing immunoassay, the irreversible antibody (refer to FIG. 2, 20E7) which is not detached during the washing process is absolutely preferred to the reversible antibody (1B5) which is easily detached. This is because the signal is generated in proportion to the concentration of the analyte from the binding body of the antigen and the antibody which is remained in solid phase after the washing. In the above assay system, it is difficult to recycle the antibody, and it is basically impossible to perform continuous measurement. However, if the reversible antibody 1B5 of which the association and dissociation can be rapidly made in the kinetic equilibrium state with the concentration of the analyte in the sample is used, it is possible to continuously recycle the antibody, so that it is possible to continuously monitor the analyte.

In addition, whether or not to implement continuous measurement according to the difference in the antibody reaction characteristic at the time of immunoassay can be further clarified through cyclic repeated measurement (refer to FIG. 3). In the case of the irreversible antibody (refer to FIG. 3, 20E7; kₐ=1.10×10⁴ Lmol⁻¹ sec⁻¹, k_{d}=1.80×10⁻⁷ sec⁻¹), after the antibody is supplied, the antibody exhibits continual attachment reaction with the fixed antigen for a predetermined time interval, and at the time of washing, the detachment is not completed. Therefore, the binding body in the antigen-antibody reaction is gradually accumulated according to the cyclic repetition. On the contrary, in the case of the reversible antibody (1B5; kₐ=4.13×10⁶ Lmol⁻¹ sec⁻¹, k_{d}=3.61×10⁻³ sec⁻¹), after the antibody is supplied, the signal is rapidly increased to reach the attachment reaction equilibrium, and at the time of washing, the detachment is immediately completed, so that the signal returns to an initial base line. The signal pattern of the attachment-detachment reversible reaction exhibits high reproducibility at the time of cyclic repetition.

Since the reversible antibody has a high dissociation rate of the binding body of the antigen and antibody, the sensitivity of assay may be deteriorated according to a decrease in affinity (equilibrium association constant K_{A}). However, if both of the association and dissociation rates are high, the affinity is not influenced. Actually, since (equilibrium association constant K_{A})=(association rate constant kₐ)/(dissociation rate constant k_{d}), if the antibody having appropriate reaction kinetics characteristics, that is, high association and dissociation rate constants is selected according to the aforementioned method, a high sensitivity of assay can be maintained. Therefore, in general, the antibody satisfying the condition of K_{A}>1×10⁸ Lmol⁻¹ is required in order to maintain a high sensitivity, and a reversible antibody having a high affinity can be defined as an antibody having characteristics of kₐ>1×10⁵ Lmol⁻¹ sec⁻¹ and k_{d}>1×10⁻³ sec⁻¹.

On the other hand, as another method of testing the affinity of the reversible antibody, the antibody is continuously diluted with the standard concentration, and the antibody is allowed to react with antigen fixed on the surface plasmon resonance sensor, and the minimum concentration of the antibody where the signal can be detected is determined, so that the affinity of the antibody can be estimated (refer to FIG. 4). Particularly, even in the case where the concentration range of the reversible antibody 1B5 is of pg/mL or less, the reversible antibody 1B5 is measured to react with the antigen. This result exhibits that the antibody has a very high affinity in comparison with the case of the existing irreversible antibody. Furthermore, it can be understood from the result of FIG. 4 that the disclosed reversible antibody reacts with the fixed antigen at a different equilibrium state in a relatively wide concentration range, so that the present invention is very suitable for manufacturing the bio sensor.

In the real-time continuous detection device according to the present invention, the reversible capturing recognizing component 10 is an antibody, a receptor, a nucleic acid, an enzyme, an aptamer, a peptide, or a molecular printing artificial membrane which can specifically bind to the analyte 11 in the sample such as living organism metabolites, a protein, a hormone, a nucleic acid, a cell, a food test material, an environment contaminant, or national-defense chemical, biological and radiological test materials.

In the real-time continuous detection device according to the present invention, the sensor may be a label-free sensor 12 (refer to FIG. 1 (A)) which directly detects the signal generated from the binding body of the analyte 11 and the capturing recognizing component 10 or a label sensor 15 (refer to FIG. 1 (B)) which performs detection through a label material 14 generating the signal in proportion to a density of the binding body of the analyte 11 and the capturing recognizing component 10. In the present invention, the label-free sensor measures a change in mass, resistance of vibrators, charge distribution, surface deformation, energy transfer, or the like on the sensor which is changed in proportion to the binding body of the analyte and the capturing recognizing component as the signal. A surface plasmon resonance (SPR) sensor which detects a difference in reflective index of light according to a change in mass of the binding body on the surface of the sensor (Robert Karlsson et al., Journal of Immunological Methods, Vol. 145, Page 229-240, 1990), a cantilever sensor which detects resistance or charge distribution of vibrators (Hans-Jurgen Butt, Journal of Colloid and Interface Science, Vol. 180, Page 251-260, 1996), an optical waveguide (evanescent) sensor (R. G. Eenink et al., Analytica Chemica Acta, Vol. 238, Page 317-321, 1990), a nanosensor using a nano-scale line or gap (Fengli Qu et al., Biosensors and Bioelectronics, Vol. 22, Page 1749-1755, 2007), or the like may be used as the label-free sensor.

In addition, in the case of using the label sensor, a detecting recognizing component labeled with the a label material is additionally reacted in order to generate a signal in proportion to the binding body of the analyte and the capturing recognizing component, and after that, the label sensor detects the signal from the label material. In the present invention, the detecting recognizing component denotes a material which can specifically bind to an analyte and be physically or chemically bound with a label material so as to detect the analyte. Herein, in the molecular level, the position of the analyte reacting with the detecting recognizing component is different from the position of the analyte reacting with the capturing recognizing component, so that the two components can simultaneously react with the analyte. As a label material which generates the signal, there are a fluorescent material, a luminescent material, an enzyme, a metal particle, a plastic particle, a magnetic particle, and the like. The sensors which sense fluorescence, luminescence, color, electro-chemical properties, magnetic field, or the like can be used as a label sensor.

In other words, in the case of using the label-free sensor, the analyte in the sample is continuously flown through a fluid channel into to a system to react with the capturing recognizing component, and in the case of using the label sensor, after the analyte in the sample reacts with the detecting recognizing component bound with the label material in advance, the analyte is continuously flown through the fluid channel into to the system to react with the capturing recognizing component.

In the real-time continuous detection device according to the present invention, the sample assay site is partitioned by a semi-permeable membrane 16 which can selectively permeate only the analyte 11 so that a recognizing reaction cell 17 is formed to the side of the surface of the sensor where the capturing recognizing component 10 is fixed.

In the real-time continuous detection device according to the present invention, in the case of using the label sensor 15, a detecting recognizing component 13 which is bound with the label material 14, which cannot permeate through the semi-permeable membrane 16 in size, is confined in the recognizing reaction cell 17 so as to be recycled.

In addition, in the real-time continuous detection device according to the present invention, the detecting recognizing component 13 and the capturing recognizing component 17 in the recognizing reaction cell 17 have reversible reaction characteristics so as to be continuously recycled.

More specifically, the sample assay site is partitioned by the semi-permeable membrane to the side of the surface of the sensor where the capturing recognizing component is fixed so that the recognizing reaction cell can be formed (refer to FIGS. 1 (C) and (D)). The small-sized analyte included in the sample permeates through the semi-permeable membrane to be diffused and transferred into the recognizing reaction cell. However, the large-sized impurity is filtrated, so that the surface of the sensor can be prevented from being contaminated. Particularly, in case of the label type assay system (refer to FIG. 1 (D)), the configuration of the recognizing reaction cell also has an effect of confining the large-sized label material bound with the detecting recognizing component in the recognizing reaction cell and recycling the label material.

According to another aspect of the present invention, there is provided a real-time continuous detection method for detecting an analyte using the aforementioned real-time continuous detection device, including the following steps: (a) injecting the sample containing the analyte through the sample inflow channel into the sample assay site; (b) binding the analyte with the reversible capturing recognizing component in the sample assay site; (c) detecting the signal generated from the binding body of the analyte and the capturing recognizing component by using the sensor; (d) detaching the analyte from the capturing recognizing component and discharging the analyte through the sample outflow channel by a continues inflow of the sample or an inflow of a washing solution; and (e) repeating the steps (b) to (d) by recycling the detached capturing recognizing component, so that a change in concentration of the analyte in the sample is measured in real time.

In the real-time continuous detection method according to the present invention, in the step (c), the signal generated from the binding body of the analyte and the capturing recognizing component is directly detected by using a label-free sensor, or the signal is measured through a label material generating the signal in proportion to a density of the binding body of the analyte and the capturing recognizing component by using a label sensor.

In the real-time continuous detection method according to the present invention, in the case of using the label-free sensor, the analyte included in the sample is continuously flown through the sample inflow channel into the sample assay site to react with the capturing recognizing component.

In the real-time continuous detection method according to the present invention, in the case of using the label sensor, after the analyte in the sample reacts with the detecting recognizing component bound with the label material in advance, the analyte is continuously flown through the sample inflow channel into the sample assay site to react with the capturing recognizing component (continuous flow exposure type), or after the analyte is continuously flown through the sample inflow channel into the sample assay site, the analyte reacts with the capturing recognizing component and the detecting recognizing component bound with the label material in the recognizing reaction cell (recognizing reaction cell type).

In the real-time continuous detection method according to the present invention, in the case of the continuous flow exposure type, the detecting recognizing component that reacts with the analyte in advance has an irreversible reaction characteristic with high binding stability, and in the case of the recognizing reaction cell type, the detecting recognizing component has a reversible reaction characteristic so that the capturing recognizing component and the detecting recognizing component can be continuously recycled.

In the real-time continuous detection method according to the present invention, in the case of using the recognizing reaction cell type label sensor, the recognizing reaction can be performed in liquid state without fixation of the capturing recognizing component on the surface of the sensor by using a principle that a fluorescence signal is generated due to interference to energy transfer between neighboring fluorescence material (label material) and fluorescence energy receptor by reaction of the capturing recognizing component and the analyte, or by using an enzyme, of which the activity is known to be suppressed by the binding of the capturing recognizing component and the analyte fixed on the enzyme molecule (label material), as the label material.

According to still another aspect of the present invention, there is provided a method of selecting a reversible capturing recognizing component used for the aforementioned real-time continuous detection device, including the following steps: (a) preparing the capturing recognizing component; (b) binding the capturing recognizing component with the analyte fixed on the surface of the sensor; (c) detecting the signal generated from the binding body of the capturing recognizing component and the analyte by using the sensor; (d) detaching the analyte from the capturing recognizing component by an inflow of a washing solution; (e) detecting a signal generated from the binding body of the capturing recognizing component and the analyte remained after the detaching by the sensor; and (f) selecting the capturing recognizing component of which the signal detected in the step (e) is lower than the signal detected in the step (c).

In the method of selecting the reversible capturing recognizing component according to the present invention, the sensor is a label-free sensor selected from a surface plasmon resonance sensor, a cantilever sensor, an optical waveguide sensor, an optical interference sensor, and a nanosensor.

In the method of selecting the reversible capturing recognizing component according to the present invention, the capturing recognizing component has a reversible reaction characteristic so that an association rate constant kₐ is in a range of from 1×10⁵ Lmol⁻¹ sec⁻¹ to 1×10⁸ Lmol⁻¹ sec⁻¹ and a dissociation rate constant k_{d} is in a range of from 1×10⁻³ sec⁻¹ to 1×10⁻¹ sec⁻¹ and a high affinity so that an equilibrium association constant K_{A}=kₐ/k_{d} is 1×10⁸ L/mol or more at the time of reacting with the analyte in the sample.

In the method of selecting the reversible capturing recognizing component according to the present invention, in the step (a), the capturing recognizing component is diluted with a carrier solution and continuously injected, and in the step (f), the capturing recognizing component generating the signal pattern, where the signal is increased and then decreased as the time elapses, is selected.

In the method of selecting the reversible capturing recognizing component according to the present invention, in the step (a), an alternative injection of the capturing recognizing component and a washing solution is repeated, and in the step (f), the capturing recognizing component generating the signal pattern, where the signal is increased and then returns to an initial base line repeatedly as the time elapses, is selected.

In a real-time detection device for detecting an analyte, a real-time continuous detection method for detecting an analyte using the real-time detection device, and a method of selecting a reversible capturing recognizing component used for the real-time detection device according to the present invention, the following advantages can be obtained.

According to the present invention, if the antibody which rapidly reversibly reacts according to a concentration of the analyte is recycled for manufacturing a bio sensor or a bio chip, configurations and manufacturing methods can be efficiently simplified in comparison with existing disposable diagnosis chip. Therefore, the number of valves and pumps required for supplying and removing reagents in an existing device or system can be minimized, so that it is possible to implement a small-sized micro flow type continuous diagnosis system which can be actually put on a human body.

According to such a new concept of a detection method (or a diagnosis scheme), diseases or symptoms can be monitored in real time, so that it is possible to solve the limitation of the disposable performance of almost all the existing immunoassay systems where the used one has to be discarded, and it is possible to continuously monitor patients of chronic diseases or high risk group patients. Furthermore, in the current diagnosis system, a long time is taken to obtain a diagnosis result and the data of monitored states of patients need to be analyzed in a laboratory. Therefore, there is a long time interval between the time of diagnosis and the time of obtaining the result of diagnosis. However, according to the present invention, it is possible to solve the problem of the current diagnosis system, where it is difficult to perform accurate diagnosis of disease or to practice a timely treatment.

Therefore, the real-time continuous detection device and the real-time detection method according to the present invention is a new preventive medicine method based on early diagnosis concept, which can satisfy the change of the clinical paradigm from the hospital-concentrated clinical service to the user-concentrated clinical service and can develop and commercialize a continuous diagnosis device capable of monitoring chronic patients and high risk group patients such as old persons always in real time. Particularly, an increase in mean life span and a decrease in fertility rate accelerate advent of an aging society, and westernization of eating habit pattern increases in various chronic adult diseases. Therefore, according to the present invention, healthy life can be maintained through early diagnosis. Particularly, the continuous diagnosis method will be applied as an original technology in the future U-health care age where a diagnosis system is installed in a mobile phone, a hospital, a house, or the like or put on a human body to measure and diagnose biological information in real time.

In addition, the real-time continuous detection device and method according to the present invention can be used to detect or assay living organism metabolites, a protein, a hormone, a nucleic acid, a cell, a food test material, an environment contaminant, national-defense chemical, biological and radiological test materials, or the like. The industrial fields and product groups associated with the present invention are as follows. In the medical diagnosis industry, there are continuous diagnosis system products for high risk group patients (chronic patients, old persons, critically ill patients), continuous infection diagnosis system products for diabetic patients, continuous relapse monitoring system products for cardiovascular patients continuous relapse monitoring system products for cancer treatment patients, health monitoring system product of closestools, or the like. In the artificial organ industry, there are artificial organ control system products such as artificial pancreas control system products. In the public health and national defense industries, there are continuous detecting system products for biological terror agents, continuous detecting system products for zoonotic infection such as avian influenza and SARS virus, and the like. In the environment industry, there are continuous monitoring system products for contamination of river, coast, sea, or the like. In the biological and food industries, there are continuous monitoring system products for biological process, continuous monitoring system products for food producing process, and the like.

### ADVANTAGEOUS EFFECTS

According to the present invention, it is possible to measure a change in concentration of the analyte by continuously recycling a predetermined amount of the reversible capturing recognizing component. The real-time continuous detection device can be used to detect or assay living organism metabolites, a protein, a hormone, a nucleic acid, a cell, a food test material, an environment contaminant, national-defense chemical, biological and radiological test materials, or the like. Accordingly, the real-time continuous detection device can be applied to medical, public health, national defense, environment, food, veterinary, biotechnology industry.

### DESCRIPTION OF DRAWINGS

FIG. 1 is a diagrammatic view illustrating (A) a continuous flow exposure type label-free sensor, (B) a continuous flow exposure type label sensor, (C) a recognizing reaction cell type label-free sensor, and (D) a recognizing reaction cell type label sensor which measures a change in concentration of an analyte by using and continuously recycling a capturing recognizing component 10 in a sample assay site according to the present invention.

FIG. 2 is a view illustrating graphs of association and dissociation reaction characteristics of a reversible antibody 1B5 and a typical irreversible antibody 20E7 produced from mouse hybridoma clone as an example of the capturing recognizing component according to the present invention, which are measured by a surface plasmon resonance sensor system where an antigen, that is, an analyte (for example, α2-macroglobulin) is fixed on a surface of a sensor, and illustrating comparisons of association and dissociation rate constants and association equilibrium constants determined from the measurement.

FIG. 3 is a graph illustrating comparisons of results of cyclic repeated measurement for testing whether or not the continuous measurement can be implemented according to a difference between the reaction characteristics of two antibodies 1B5 and 20E7 by using the surface plasmon resonance sensor system of FIG. 2.

FIG. 4 is a view illustrating results of test of the affinity of the reversible antibody 1B5 with respect to the antigen, which are obtained through reaction of the antibody which is continuously diluted and the antigen fixed on the sensor according to a change in concentration of the antibody.

FIG. 5 is a view illustrating comparison of results of evaluation whether or not the reversible antibody 1B5 can be used for medical clinical diagnosis by allowing an antigen, that is, an analyte react with the reversible antibody 1B5 fixed on a surface of a sensor according to an increase in concentration of the analyte and by using (A) a phosphate buffer solution and (B) a human serum as a sample carrier solution.

FIG. 6 is a view illustrating results of signal amplification obtained by additionally introducing a polymer between a gold colloid particle having a diameter of 30 nm as the label material 14 and an irreversible antibody 20E7 as the detecting recognizing component 13 in order to improve the sensitivity of assay of the sensor system illustrated in FIG. 5.

FIG. 7 is a view illustrating results of response of a sensor according to a change in concentration of the analyte under the conditions that the micro flow rate into the sensor chip is lower by 1/10 times than that of the former experiment condition in order to minimize the sample consumption by using the sensor system illustrated in FIG. 5.

FIG. 8 is a view illustrating (A) results of concentration response and (B) a graph depicting its standard curve, obtained from SPR signal of the sensor according to a change in concentration of the analyte (α2-macroglobulin), which is continuously increased and decreased by 10 times in two cycle repetitions, by operating a surface plasmon resonance sensor system where the reversible antibody 1B5 is fixed on the surface of the sensor in a continuous measurement mode in order to exemplify the recycling of the reversible antibody.

FIG. 9 a view illustrating results of concentration response of the sensor in the sensor system illustrated in FIG. 8 according to an arithmetic change in concentration where the concentration of the analyte is increased and decreased by two times or less.

### REFERENCE NUMERALS

10: capturing recognizing component
11: analyte
12: label-free sensor
13: detecting recognizing component
14: label material
15: label sensor
16: semi-permeable membrane
17: recognizing reaction cell

### BEST MODE

Hereinafter, a real-time continuous detection device using a reversible capturing recognizing component according to the present invention will be described in detail.

### (1) Example of Configuration of Real-Time Continuous Detection Device Using Label-Free Sensor

In addition to a reversible recognizing component, a sensor technology is one of the essential factors for configuring a real-time continuous detection device (or a real-time continuous detection system). As described above, sensors may be mainly classified into to a label-free sensor and a label sensor. Theoretically, for simplifying the configuration of a continuous diagnosis system, a label-free sensor such as a plasmon resonance sensor, a cantilever sensor, or an optical waveguide sensor may be used.

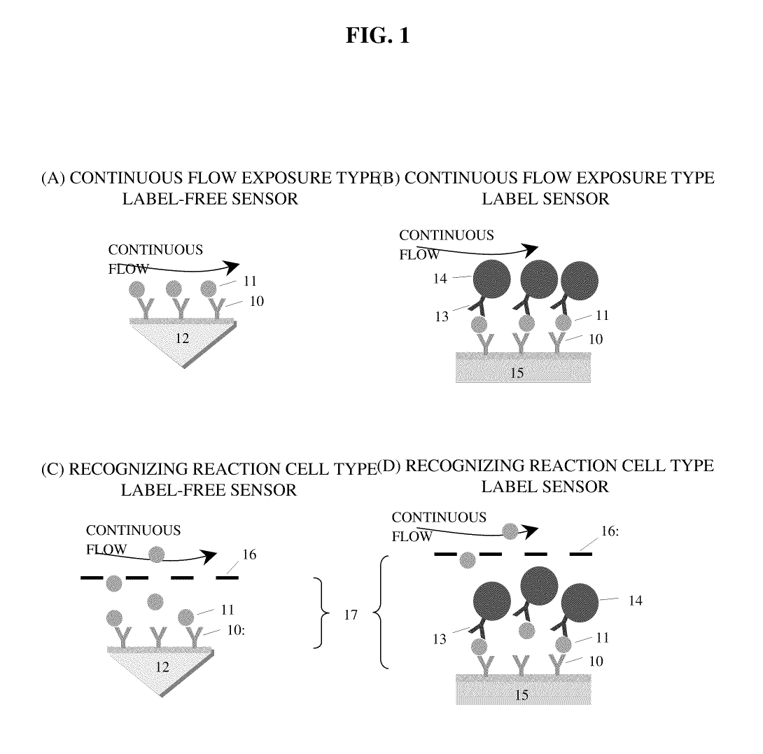

There are various configurations of the continuous detection device. For the convenience of description of the usability of the present invention, a label-free sensor-based continuous detection device where a reversible antibody 1B5 is fixed on a plasmon resonance sensor chip is exemplified with reference to FIG. 1, as follows.

As a representative method using a label-free sensor, there is a method of measuring surface plasmon resonance which is a charge density wavelength generated from light in an interface between a metal and a dielectric medium. The surface plasmon resonance interacts with a material in an area very close to the surface of the metal. Therefore, due to recognizing reaction or the like in the area, a change in an optical characteristic influences the incident angle of light inducing the surface plasmon resonance (J. Homola et al., Sens. Actuators B, Vol. 54, Page 3-15, 1999). Accordingly, a change in the incident angle of the light inducing the surface plasmon resonance caused by a reaction between an analyte and a recognizing component on the surface of the sensor is measured as a signal.

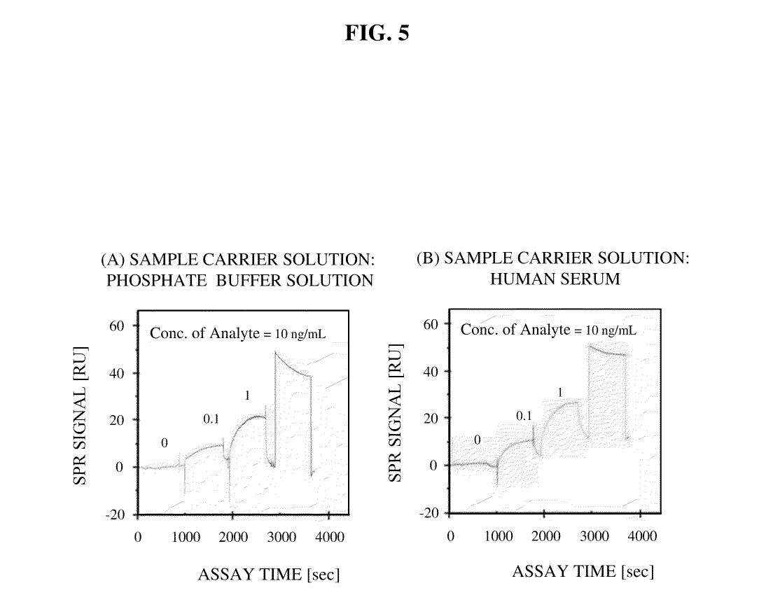

A continuous detection device (refer to (A) of FIG. 1) is configured so that a reversible antibody (1B5) 10 is fixed on a surface plasmon resonance sensor 12, and standard solutions are produced so as to contain analytes (α2-macroglobulin) having different concentrations by diluting with a phosphate buffer solution. While the standard solutions are sequentially injected into the continuous detection device at a micro flow rate of 10 μL/min, response signals in proportion to the concentration are generated from the sensor (refer to Embodiment 6). Herein, each of the standard solutions is injected after the signal is allowed to return to the base line. Under given conditions, the sensitivity of measurement is high (0.1 ng/mL or less), and the concentration response time is short (640 seconds with 95% of the final response level as a reference) (refer to (A) of FIG. 5). In order to test the assay specificity, analytes having the same concentration range are produced by diluting with human serum as a medical clinical sample, and the above experiment is repeated. As a result, substantially the same concentration response is obtained (refer to (B) of FIG. 5). Accordingly, it can be understood that the aforementioned continuous detection device can be used for medical clinical diagnosis.

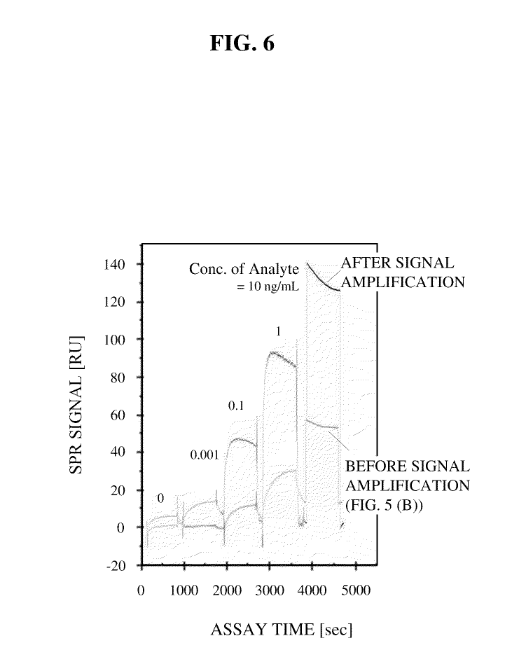

In addition, as a method of improving the sensitivity of assay, there may be used a signal amplification method where a detecting recognizing component 13 bound with a label material 14 is additionally introduced and a mass of a binding body of an analyte 11 and a capturing recognizing component 10 in the recognizing reaction is increased. The irreversible antibody 20E7 is selected as the detecting recognizing component 13, and the detecting recognizing component 13 is physically bound with a gold colloid particle having a diameter of 30 nm. The binding body is allowed to react with the standard solution of the analyte in advance. While the reacted product is injected into the sensor, the concentration response of the sensor is measured (refer to Embodiment 7). The irreversible antibody 20E7 together with the reversible antibody 1B5 can react with the analyte. As a result, if the signal amplification method is used, the minimum of 0.001 ng/mL of the analyte can be sensed, so that the sensitivity of assay can be improved by 100 times (refer to FIG. 6).

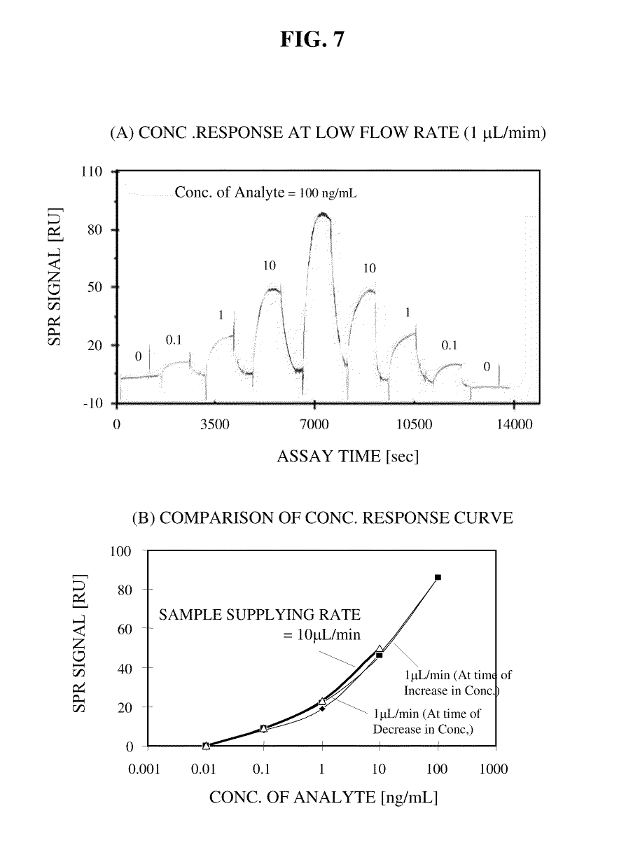

Particularly, in the case of medical clinical sample, the sample consumption needs to be minimized, so that the micro flow rate is set to be decreased down to 1/10 times the former flow rate (that is, 1 μL/min or 1.44 mL/day). Under the same conditions, the response of the sensor is measured according to a change in concentration of the analyte (refer to Embodiment 8). As the result of measurement of the concentration response of the sensor, the sensitivity of assay (0.1 ng/mL) and response time (640 seconds, with 95% of the final response as a reference) are maintained constant regardless of the micro flow rate (refer to (A) of FIG. 7). In addition, there is no great change in the pattern of the concentration response of the sensor according to the change in the micro flow rate, the two concentration response curves that are obtained under the different conditions where the flow rates are different by 10 times are substantially coincident with each other (refer to (B) of FIG. 7). In addition, there is also no difference between the concentration response measured at the time when the concentration of the analyte is increased and the concentration response measured at the time when the concentration thereof is decreased.

As illustrated in FIGS. 5 to 7, in order to obtain the response of the SPR sensor according to a change in concentration of the analyte by using the sensor chip where the reversible antibody is fixed, a “reset mode” is used. In the reset mode, the measurement starts after the device is allowed to return to the initial condition, that is, the original state where there are no analyte every time when the concentration is changed.

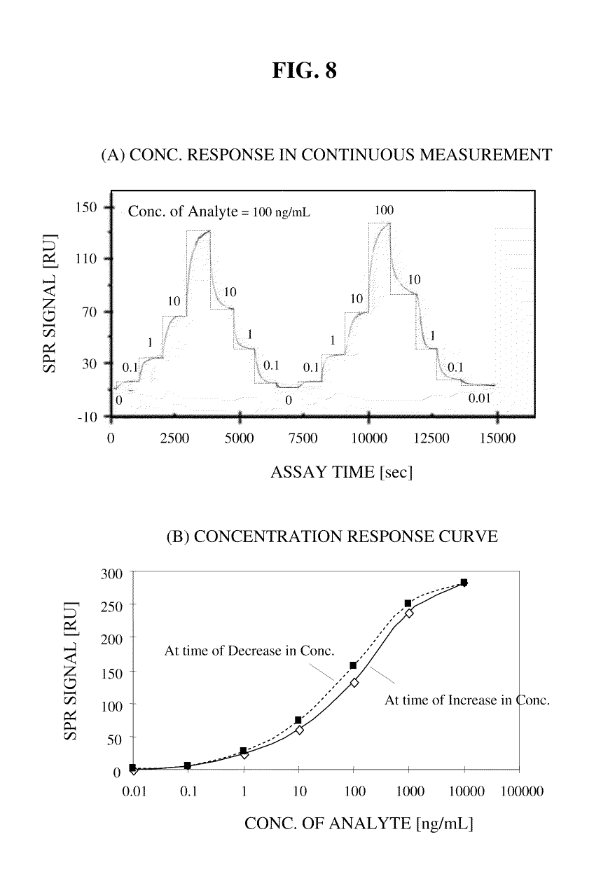

In order to exemplify the recycling of the reversible antibody, the “continuous mode” is used. In the continuous mode, the concentration of the analyte is increased and decreased stepwise by 10 times every 15 minutes (in a range of from 0.01 ng/mL to 100 ng/mL), the concentration response of the sensor is continuously obtained for twice repetition of the change (refer to Embodiment 9). The concentration response of the sensor reaches an equilibrium state within 15 minutes at the changed concentration of the analyte that is injected into the sensor at the given micro flow rate (1 μL/min), and high reproducibility is exhibited in twice repetition (refer to FIG. 8 (A)). The standard curve (refer to FIG. 8 (B)) representing the concentration response of the sensor measured in the continuous measurement mode is somewhat different from the curve measured in the reset mode. It is determined that this difference is caused from a difference in operation scheme of the sensor system.

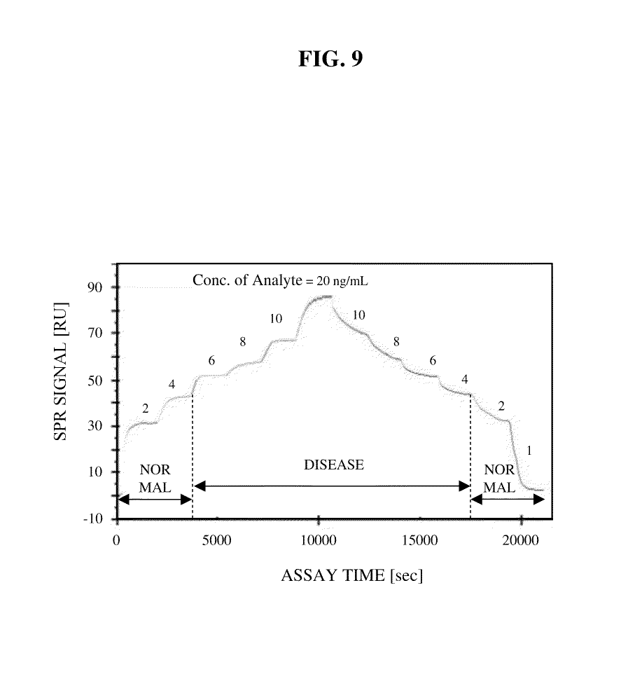

Since the changing patterns of the concentration at the time of occurrence of disease or symptom may be different according to the type of the analyte, the concentration response of the sensor according to the arithmetic change in concentration, which is increased or decreased by twice or less, is measured in the continuous mode (refer to Embodiment 10). Similarly to the concentration response according to the exponential change in concentration, the sensor also exhibits similar assay performance with respect to the arithmetic change in concentration of the analyte (refer to FIG. 9). Furthermore, since the sensor responds very sensitively and rapidly with respect to a very small change in concentration, it is expected that the reversible antibody-based bio sensor will be widely applied to measure analytes requiring very accurate assay in the future

In the present invention, as the analyte for exemplifying the continuous diagnosis, α2-macroglobulin is selected. A reversible antibody specific to the analyte is produced, and the continuous diagnosis method is exemplified. The macroglobulin may be used as bio markers of three types of diseases. In other words, the macroglobulin may be used for checking the treatment and relapse of a nephrotic syndrome, early diagnosis of Alzheimer's disease, and clinical diagnosis of inflammation reaction and complicating disease after artificial organ transplantation.

In addition, about 90% cases of the nephrotic syndrome occur in infants. The nephrotic syndrome is a renal disease where protein is contained in urine. The protein is leaked due to abnormality of glomerulonephritis of the nephron (Daniel A. Blaustein et al., Primary Care Update for OB/GYNS, Vol. 2, Page 204-206, 1995). In most cases, edema occurs in patient's body or legs. In some cases, the nephrotic syndrome proceeds to a nephrosclerosis, a renal failure syndrome, or a cancer. The diagnosis of this disease is performed by CBC (complete blood count), liver function test, nephron function test, blood protein (macroglobulin or the like) test, urine test, or the like. If the nephrotic syndrome is found, an immunosuppressant (prednisone) or a steroid medicine is medicated for one to six months as treatment. During this time interval, urine test or blood test is repeatedly performed, and the change is observed, so that the treatment effect is checked. For precise observation, the patient needs to periodically go to hospital, and the blood test and the urine test needs to be performed. In particular, since 90% of the nephrotic syndrome patient is infants who have weak ability to express themselves, more precise test and observation for a change of the symptoms and an abnormality of the body need to be repeatedly performed. Therefore, it is expected that the development of the continuous detecting method for the blood protein such as a macroglobulin will be useful as a further technology of checking the treatment effect and relapse of the nephrotic syndrome of infants.

Another example of using the macroglobulin as a bio marker is Alzheimer's disease. The disease occurs in one of 60˜70 persons. The disease is the geriatric disease that 50% of old persons of 85 years or more suffer from. Therefore, the disease needs to be prevented through early diagnosis. In 2006, a research team of London King's College found out from blood test that the concentrations of two types of protein, that is, a precursor of complementary factor H and α2-macroglobulin are increased in the patient having the Alzheimer's disease. Therefore, due to the checking of the disease using the difference in the concentration of the protein, the early diagnosis of the disease can be performed (A. Hye et al., Brain, Vol. 129, Page 3042-3050, 2006). If the Alzheimer's disease is early diagnosed by the continuous detecting of the macroglobulin, the disease can be prevented and the treatment is early made and the proceeding of the disease can be further slowed down in comparison with the case where the diagnosis is performed at the hospital after the occurrence of the symptom. Therefore, this technology is expected to improve the quality of life.

Still another example of using the macroglobulin as a bio marker is diagnosis of an inflammation reaction or a complicating disease associated with artificial organ transplantation. There were not so many research results of the markers for the diagnosis index. In 2005, Medical Center of Duke University in USA disclosed a research result that the concentration in α2-macroglobulin is increased by 50% in the case a cardiopulmonary bypass machine is used in a heart surgery (Eric A. Williams et al., J Thorac Cardiovasc Surg, Vol. 129, Page 1098-1103, 2005). This result indicates that the change in the macroglobulin can be used as an index of a systemic inflammation reaction. Therefore, if the bio marker for the occurrence of the inflammation reaction can be continuously detected at the time of prognostic observation after the artificial organ transplantation, there is an advantage in that the replacement of artificial device or the treatment of the complicating disease can be early performed in comparison with the case of the periodical treatment at the hospital or the treatment after the occurrence of the complicating disease.

In general, the aforementioned diseases and other acute and chronic diseases relatively slowly proceed in units of time or day. Therefore, the response time of the sensor for measuring the bio marker as an index of the disease is required to be typically in units of minute. If the response time of the sensor is shorter 10 times than the proceeding time of the disease, the proceeding of the disease becomes the rate controlling step in the process of continuously diagnosing the bio marker. Therefore, the concentration response time (about 15 minutes with 95% of the final response as a reference) of the sensor with respect to the macroglobulin illustrated in FIGS. 8 and 9 satisfies the continuous detecting condition. A shorter response time (for example, in units of second) of the sensor does not influence the assay performance in the continuous diagnosis. On the other hand, a disposable sensor (for example, a blood glucose sensor) having a different concept has only the effect that only the measurement time for the sample is shortened.

### (2) Example of Construction of Diagnosis System Using Label Sensor

In many examples of the label sensor, a fluorescent material is used as a signal generating source. A capturing recognizing component 10 fixed on a surface of a solid phase can be used (refer to FIGS. 1 (B) and (D)), or a liquid phase reaction in a recognizing reaction cell 17 can be performed for detection. In this case, a principle is used where light emitted from the fluorescent material (donor) that is the signal generating source is absorbed by an energy receptor (acceptor) which is very close to the light and no light is extremely emitted (Shaw et al., J. Clin. Pathol, Vol. 30, Page 526-531, 1977). As an application thereof, a recognizing reaction such as an antigen-antibody attachment reaction may be designed so as to control energy transfer between the fluorescent material and the energy receptor, and the fluorescence signal is detected by a light-receiving device (a photodiode, a charge-coupled device, a photomultiplier tube, or the like).

In another example of the label sensor, an enzyme may be used as a label material. The capturing recognizing component 10 fixed on a surface of a sensor can be used (refer to FIGS. 1 (B) and (D)), or a liquid phase reaction in the recognizing reaction cell 17 can be performed if several types of enzymes, of which the activity is suppressed by attaching an antibody on the enzyme molecule, are used. The principle that the binding between the enzyme and the analyte (that is, an antigen) suppresses the activity of the enzyme can be used for immunoassay (Se-Hwan Paek et al., Biotechnology and bioengineering, Vol. 56, Page 221-231, 1997). The signal from the enzyme can be measured by an absorbance measurement sensor (a spectro-photometer), a light-receiving sensor (a photodiode, a charge-coupled device, a photomultiplier tube, or other light-receiving devices), an electro-chemical sensor (electrode), or other various means according to the type of the selected enzyme and substrate.

In still another example of the label sensor, a magnetic particle may be used as a label material. If the capturing recognizing component 10 fixed on the surface of the sensor is used (refer to FIGS. 1 (B) and (D)), the magnetic field formed according to the reaction between an analyte and the recognizing component can be measured (A. Perrin et al., Journal of Immunological Methods, Vol. 224, Page 77-87, 1999). As representative magnetic field measurement sensors, there are GMR/TMR devices and Hall devices, which has low power consumption and small size and light weight and which can be integrated.

As described hereinbefore, in a continuous diagnosis system constructed by combining a reversible antibody and a sensor technology, the antibody can be continuously recycled without the sacrifice of the sensitivity of assay, and the analyte can be measured in real time. Since the concentration response time is tens of minutes with 95% of the final response as a reference, the exemplified continuous diagnosis system can be used applied to measure the analyte of which the concentration is changed in units of minute or more. In particular, the exemplified continuous diagnosis system is suitable for an assay object requiring an alarm when the concentration exceeds a predetermined upper limit. As applicable fields of the continuous diagnosis system, there are continuous diagnosis of disease or symptom, control of artificial organ, continuous detecting of biological terror agent, continuous monitoring of environment contaminant, and continuous monitoring of biological process.

### MODE FOR INVENTION

Hereinafter, embodiments of the present invention will be described in detail. Since these embodiments are provided in order to exemplify the present invention, the scope of the present invention is not limited to these embodiments.

### Experiment Resources

The materials and purchasing sites thereof used for the embodiments of the present invention are as follows. A surface plasmon resonance sensor chip (BIACORE CM5; components: a glass maternal part, a gold thin film having a thickness of 30 nm, and a dextran layer having a thickness of 100 nm), an amine coupling kit (including 100 mM N-hydroxysuccinimide (NHS), 400 mM N-ethyl-N′-(dimethylaminopropyl)carbodiimide) (EDC), 1M ethanolamine hydrochloride, pH 8.5), and 40% glycerol are purchased from GE healthcare (Sweden). A mouse monoclonal antibody (20E7, 3D1; irreversible reaction characteristic) and α2-macroglobulin (tetramer) are supplied from Ab Frontier (Korea). A bovine serum albumin, sodium acetate, sodium phosphate, sodium chloride, glycine, human AB serum (human serum, AB plasma), casein, gold nanoparticle (30 nm), a polymer of a goat anti-mouse antibody and horseradish peroxidase (HRP), and 3,3′,5,5′-tetramethylbenzidine (TMB) are purchased from Sigma (USA). A total IgG antibody quantitative kit (mouse IgG core ELISA) is supplied from Corma Biotech (Korea). With respect to other reagents, assay-class reagents were used.

### Embodiment 1

### Production of Reversible Antibody of Mouse Monoclonal

A hybridoma cell producing the monoclonal antibody is manufactured according to a typical standard method. More specifically, an α2-macroglobulin as an immunogen is injected into an abdominal cavity of a female BALB/c mouse which is 6 weeks old. After the immunization, boosting is performed three times in an interval of two weeks. At the third day after the third boosting, mouse is scarified, and the spleen is extracted. The obtained spleen cell is cell-fused with a myeloma cell strain (Sp2/0-Ag14). After that, the hybridoma cell is selected.

With respect to the hybridoma cell, a total of 384 type clones are produced. By using a culture solution containing the antibody produced from each clone, a test of the antibody reaction characteristic to the immunogen and a determination of the total IgG antibody amount are performed. For the test of the antibody reaction characteristic to the immunogen, each of the clone culture solutions are transferred to react in 96 micro plate wells where the α2-macroglobulin (2.5 μg/mL) diluted by 10 mM phosphate buffer solution (containing 140 mM NaCl; pH 7.4) is fixed. After washing, a polymer ( 1/5000) of goat anti-mouse antibody-HRP diluted by 10 mM phosphate buffer solution (casein-PBS) containing 0.5% casein is allowed to react. After washing again, an HRP substrate solution (0.05M acetate buffer solution (pH 5.1 (10 mL)) containing 3% hydrogen peroxide (10 μL) and 10 mg/mL TMB (100 μL; diluted by dimethyl sulfoxide as a solvent)) is added to each well, so that an enzyme reaction is performed. After 15 minutes, 2M sulfuric acid is added, and the reaction is allowed to stop. The color signal generated from each well is measured at the absorbance of 450 nm by using a micro plate reader (VERSAmax™, produced by Molecular Devices, USA). In addition, the total IgG antibody amount is determined by using the mouse IgG core ELISA kit according to the assay process provided from the manufacturer.

From the two assay results, seven types of the hybridoma clones simultaneously satisfying the conditions for the test of the antibody reaction characteristic, that is, the absorbance of 2.0 or less (lower 50%) and the condition for the total IgG antibody amount of 0.1 μg/mL or more (upper 15%) are selected.

### Embodiment 2

### Surface Activation of Sensor Chip and Fixation of Liqand on Sensor Chip

The surface of the surface plasmon resonance sensor chip BIACORE CM5 is activated by using 100 mM NHS and 400 mM EDC according to the protocol provided from the manufacturer. The amount of the ligand (an antigen or an antibody) that is to be fixed on the surface of the sensor chip is calculated and determined according to the protocol guide provided from the manufacturer. The ligand is diluted to a predetermined concentration by a buffer solution of 10 mM sodium acetate (pH 4.0). The diluted ligand is injected into the sensor chip (flowrae=5 μL/min), so that the fixation is performed. After 20 minutes, a solution of 1M ethanolamine hydrochloride (pH 8.5) is injected for 6 minutes, the remaining surface of the sensor is non-activated.

The operation of the surface plasmon resonance sensor system (BIACORE 2000, produced by GE healthcare, Sweden) is performed according to the BIACORE 2000 usage protocol provided from the manufacturer. As a sample carrier solution (running buffer), the phosphate buffer solution or the human serum is selectively used according to the purpose of test. As the sensor chip which is to be installed in the sensor system, the BIACORE CM5 is purchased. In the sensor chip, the bovine serum albumin as a control group is attached in a first fluid channel, and the ligand is chemically fixed in a second fluid channel. In all the embodiments, the flow direction is set to the direction from the first channel to the second channel, and a pure signal value is obtained by subtracting a noise value of the first channel from a signal value (resonance unit; RU) of the second channel. In all the embodiments, the internal temperature of the reaction cell is maintained to be 25° C.

### Embodiment 3

### Screening of Reversible Antibody Using Surface Plasmon Resonance Measurement System

For the purpose of screening of the reversible antibody, as described in Embodiment 2, a sensor chip is produced by fixing the bovine serum albumin at the concentration of 100 μg/mL in the first fluid channel and fixing the α2-macroglobulin at the concentration of 100 μg/mL the concentration in the second fluid channel. After the prepared sensor chip is installed in the surface plasmon resonance measurement system, injection is performed at a rate of 5 μL/min by using 10 mM phosphate buffer solution as a sample carrier solution, and an equilibrium state is maintained. The seven types of the hybridoma clones that are selected through the test of the antibody reaction characteristic and the determination of the total IgG antibody amount in Embodiment 1 are appropriately diluted by 10 mM phosphate buffer solution (PBS, pH 7.4). According to the assay program (BIACOREoperation 2000) provided from the manufacturer, each antibody sample (35 μL) is injected into the sensor chip installed in the sensor system for 420 seconds so as to induce an attachment reaction. After that, the phosphate buffer solution is injected for 210 seconds so as to induce a detachment reaction. After the completion of assay for the same type of the antibody, 10 mM glycine buffer solution (pH 1.5, 15 μL) is injected at a constant rate for 180 seconds, so that the surface of the sensor is reproduced. The patterns of the association and dissociation reactions are assayed by using an editing program (BIAevaluation 2.0) provided from the manufacturer, and an association rate constant kₐ, a dissociation rate constant k_{d}, and an equilibrium association constant K_{A} are calculated. The following Table 1 lists the association rate constants kₐ, the dissociation rate constants k_{d}, and the equilibrium association constants K_{A} of the seven types of the tested hybridoma clones.

TABLE 1
Reaction Characteristics of Secondarily Selected hybridoma clones

| | | | equilibrium |
|---|---|---|---|
| | association rate | dissociation rate | association |
| | constant (kₐ) | constant (k_{d}) | constant (K_{A}) |
| Name of Clone | L · mol⁻¹ · sec⁻¹ | sec⁻¹ | L/mol |
| 1B5 | 4.13 × 10⁶ | 3.61 × 10⁻³ | 1.14 × 10⁹ |
| 1F8 | 2.24 × 10⁶ | 3.87 × 10⁻³ | 5.79 × 10⁸ |
| A2, 1-14 | 3.74 × 10⁵ | 7.73 × 10⁻³ | 4.84 × 10⁷ |
| T4-2, 2 | 3.21 × 10⁵ | 5.41 × 10⁻² | 5.93 × 10⁵ |
| T1-12, 7 | 5.15 × 10⁴ | 1.79 × 10⁻² | 2.88 × 10⁵ |
| 23E10 | 1.31 × 10⁴ | 1.82 × 10⁻² | 7.20 × 10⁵ |
| 9A3 | 6.37 | 1.00 × 10⁻⁵ | 6.37 × 10⁵ |

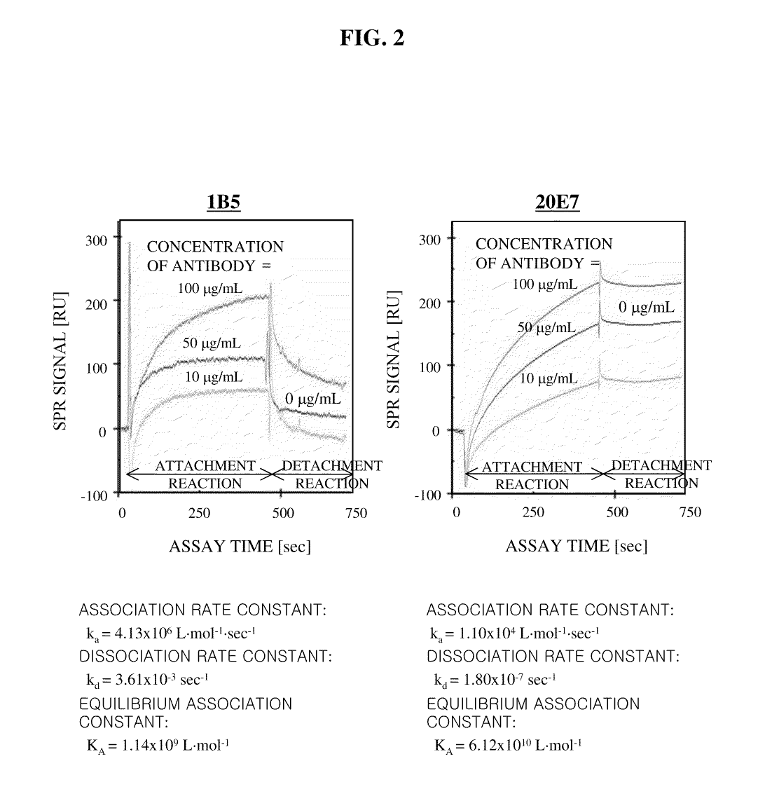

Among the seven types of the tested hybridoma clones, two clones (1B5 and 1F8) exhibit a high affinity and a reversible reaction characteristic. Since the antibody produced from the clone 1B5 exhibits high affinity of 1×10⁹ L/mol or more, the antibody is selected as one suitable for the object of the present invention. The reaction characteristics of the antibody is compared with those of a the typical irreversible antibody 20E7 (refer to FIG. 2). In the attachment reaction, the antibody 1B5 reaches the equilibrium state faster than the antibody 20E7. In the detachment reaction, the antibody 1B5 is fallen near to the initial value, but the antibody 20E7 is not almost detected. Due to the difference in the characteristic, in the existing disposable immunoassay requiring washing, the irreversible antibody 20E7 that is not detached by the washing is preferentially used. On the contrary, the antibody 1B5 of which the association and dissociation are rapidly performed through a kinetic equilibrium reaction according to the concentration of the antibody can be used for continuous measurement using the antibody recycling. Therefore, the existence of the reversible antibody as a basic material of the present invention is disclosed, and the essential difference in characteristic from the existing antibodies is primarily demonstrated. As a reference, both of the antibody 1B5 and the antibody 20E7 exhibit specific reaction characteristic with respect to the α2-macroglobulin, and the antibodies can be attached to different epitopes of the antigen molecule) so as to simultaneously react with the same antigen molecule.

### Embodiment 4

### Comparison of Patterns of Attachment/Detachment Cyclic Reaction Between Reversible Antibody and Irreversible Antibody

The pattern of the attachment/detachment cyclic reaction of the reversible antibody 1B5 is obtained in the same experimental conditions by using the sensor chip produced in Embodiment 3, and the pattern is compared with that of the irreversible antibody 20E7. The antibody solution (100 ng/mL 1B5 or 20 ng/mL 20E7; 17.5 μL) diluted by 10 mM phosphate buffer solution is injected into the sensor chip at a flow rate of 5 μL/min for 210 seconds so as to induce the attachment reaction. After that, the phosphate buffer solution is injected for 110 seconds so as to induce the detachment reaction. Under the same conditions, the association and dissociation reactions are repeated 6 times with respect to each antibody. After the completion of assay for the types of the antibodies, 10 mM glycine buffer solution (pH 1.5, 15 μL) is injected at a constant rate for 180 seconds, so that the surface of the sensor is reproduced. The operation of the measurement system BIACore 2000 and data editing are the same as those described in Embodiment 2.

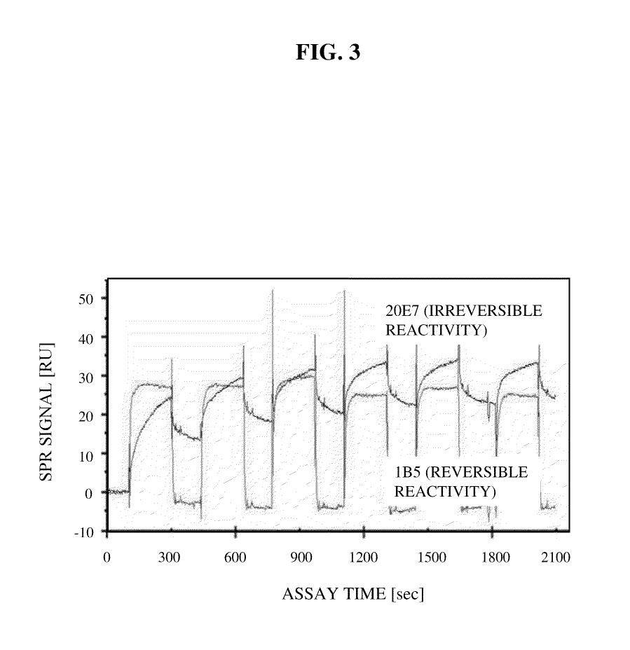

As a result of the assay, in FIG. 3, with respect to the antibody 1B5 which is expected to exhibit the reversible reaction characteristic, within one minute after the injection of the antibody, the signal is increased to reach the equilibrium state of the attachment reaction to the fixed antigen. When the phosphate buffer solution is injected, the antibody is immediately detached, so that the signal returns to the initial value. This pattern of the attachment/detachment reversible reaction exhibits high reproducibility in the case of six repetitions. With respect to the antibody 20E7 which is expected to exhibit the irreversible reaction characteristic, for a predetermined time after the injection of the antibody, the attachment reaction is continuously performed at a relatively slow rate. When the phosphate buffer solution is injected, the detachment reaction is not completed. Therefore, the binding body in the antigen-antibody reaction is gradually accumulated according to the repetition of the attachment/detachment reaction, so that the signal is increased in a stepwise pattern.

### Embodiment 5

### Determination of Lower Limit of Reaction Concentration of Reversible Antibody

The response of the surface plasmon resonance sensor according to the change in concentration of the reversible antibody 1B5 is measured by using the sensor chip produced in Embodiment 3 and the same experiment method. The antibody 1B5 is diluted to the concentration ranging from 0.5 pg/mL to 0.5 μg/mL by using 10 mM phosphate buffer solution. Each of the diluted solutions (17.5 μL) of the antibody is injected at a flow rate of 5 μL/min for 210 seconds so as to induce the attachment reaction. After the phosphate buffer solution is injected for 110 seconds so as to induce the detachment reaction. Under the same conditions, in one cycle test, assay is performed in the order of from a low concentration solution of the antibody to a high concentration solution, and after that, the assay is performed in the reverse order. After the completion of the cycle test, the surface of the sensor is reproduced according to the same method as that of Embodiment 4.

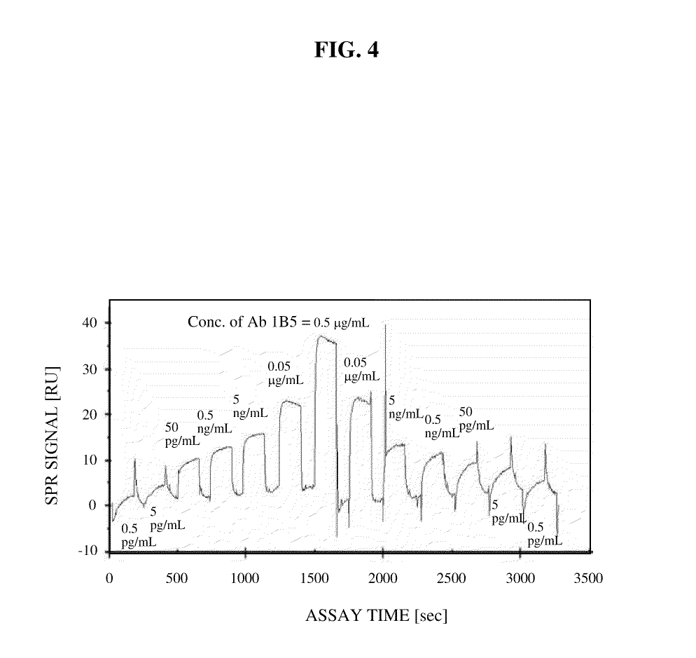

As illustrated in FIG. 4, in the concentration range of the used antibody, the signal of the surface plasmon resonance sensor is increased in proportion to the stepwise increase in concentration of the solution of the antibody, and the signal is decreased in proportion to the stepwise decrease in concentration. In particular, even in the case where the concentration range of the antibody is of pg/mL or less, the antibody is measured to react with the antigen fixed on the sensor chip. This result exhibits that the antibody has a high affinity in comparison with the irreversible antibody used for the existing immunoassay. Therefore, the immunoassay system in which the antibody having the reaction characteristics such as the antibody 1B5 is installed is expected to exhibit an excellent sensitivity of assay. In addition, since the antibody has the reaction characteristic in the concentration unit of pg, an immunosensor using the antibody which is manufactured in the future is expected to have a wide measurement range.

### Embodiment 6

### Construction of Reversible Antibody-Based Label-Free Immunosensor System

In configuration of a continuous flow exposure type label-free sensor system for measuring α2-macroglobulin by using a reversible antibody (refer to FIG. 1 (A)), the surface plasmon resonance sensor system (BIACORE 2000) and the sensor chip BIACORE CM5 where the reversible antibody is fixed are used. As described in Embodiment 2, the sensor chip is produced by fixing the bovine serum albumin at the concentration of 100 μg/mL in the first fluid channel and fixing the reversible antibody 1B5 at the concentration of 10 μg/mL the concentration in the second fluid channel. In this manner, the macroglobulin that is an analyte specifically reacting with an antibody fixed on the surface of the sensor is diluted by 10 mM phosphate buffer solution, so that a standard sample in a concentration range of from 0 to 10 ng/mL is produced. Each standard sample (150 μL) is injected at a flow rate of 10 μL/min for 900 seconds into the sensor chip installed in the sensor system so as to induce the attachment reaction. After that, a phosphate buffer solution is injected for 120 seconds so as to induce the detachment reaction. After the completion of assay with respect to each sample, similarly to Embodiment 4, the surface of the sensor is reproduced. By using human serum as a diluted solution and a sample carrier solution instead of the phosphate buffer solution, the experiment is repeated under to the same conditions as those described above.

In the immunoassay field, a reversible antibody-based assay system is firstly constructed by using the surface plasmon resonance sensor. In the assay system, the concentration response with respect to the selected analyte is obtained in a concentration range of from 0.1 to 10 ng/mL, and a lower detection limit of the concentration indicating the sensitivity of measurement is 0.1 ng/mL or less (refer to FIG. 5 (A)). In order to test the assay specificity of the assay system, the measurement is performed by using human serum as a sample carrier solution and a diluted solution for the standard sample as the conditions close to a medical clinical test (refer to FIG. 5 (B)). The result of measurement shows the concentration response similar to the case (A) using the phosphate buffer solution. Therefore, the reversible antibody (1B5)-based sensor system has excellent sensitivity of measurement and assay specificity. In addition, the reversible antibody-based sensor system can be applied to an actual medical clinical test.

### Embodiment 7

### Siqnal Amplification Usinq Detectinq Antibody Labeled with Gold Nanoparticle

### Embodiment 7.1

### Manufacturing Polymer Between Detecting Antibody and Gold Nanoparticle

A gold colloid (diameter: about 30 nm) suspension is manufactured by a standard method using sodium citrate as a reductant (L. A. Dykman, A. A. Lyakhov, V. A. Bogatyrev, S. Y. Chchyogolev. Colloid, 60, 700, 1998). More specifically, tertiary deionized water (1,000 mL) is contained in a glass flask, and 1% gold chloride solution (tetrachloroauric acid) (20 mL) is added. For facilitation of the reaction, a hot plate is used to boil the solution. In order to produce a gold colloid, 1% sodium citrate solution (40 mL) which is filtrated by using a 0.2 μm filter is added as a reductant. After the addition of the sodium citrate, the solution is changed from black to red in color. After heating for 10 minutes, the reaction is allowed to stop. The resulting product is gradually cooled at the room temperature. The resulting product is reserved in a refrigerator so as to be used for the experiments.

0.5M carbonate buffer solution (pH 9.6; 1 μL) is added to the manufactured gold nanoparticle suspension (1 mL) to adjust the pH to be about pH 8.0. The irreversible antibody 20E7 (refer to FIG. 2) diluted at a concentration of 150 μg/mL by 10 mM phosphate buffer solution (PB; containing no NaCl) (100 μL) is added to the solution. After the reaction at the room temperature for one hour, PB (casein-PB; 122 μL) containing 5% casein is added, and the reaction is performed again at the room temperature for one hour. The mixture is centrifuged at 16,000 rpm for 30 minutes. After that, the supernatant solution is removed, and the precipitate is dissolved with casein-PB (400 μL). After the resulting product is centrifuged at 16,000 rpm for 30 minutes, the supernatant solution is removed, and the precipitate is dissolved with casein-PB (50 μL) so as to be condensed by 20 times with the gold particle as a reference.

### Embodiment 7.2

### Concentration Response of Assay System Using Signal Amplification

A standard sample in a concentration range of from 0 to 10 ng/mL is manufactured by diluting the α2-macroglobulin, that is, an analyte with human serum. Before each sample is inserted into the sensor chip installed in the sensor system, the sample reacts with the polymer (10 ng/mL) of the detecting antibody and the gold nanoparticle, which is manufactured in Embodiment 7, at the room temperature for 10 minutes. The reaction mixture (150 μL) is injected into the sensor chip manufactured in Embodiment 6 at a flow rate of 10 μL/min for 900 seconds so as to induce the attachment reaction. After that, the human serum is injected at the same flow rate for 120 seconds so as to induce the detachment reaction. After the completion of assay for each sample, similarly to Embodiment 4, the surface of the sensor is reproduced. FIG. 5 illustrates that the concentration response of the assay system using the signal amplification step is improved in comparison with the concentration response of the label-free sensor system obtained in Embodiment 6. In actual cases, the sensitivity of assay is improved by 100 times from the level of 0.1 ng/mL (refer to FIG. 5 (B)) to the level of 0.001 ng/mL. Even an analyte having a very low concentration in the sample can be measured by using the signal amplification method according to an example of the present invention, so that the continuous detecting method using the reversible antibody can be widely applied to the measurement of various types of analytes.

### Embodiment 8

### Pattern of Concentration Response of Assay System According to Decrease in Flow Rate

In the case of a medical clinical sample, particularly, the using amount needs to be minimized. Therefore, the concentration response of the assay system is obtained by using the micro flow rate which is decreased by 1/10 times that of the former experiment condition. The same sensor chip as that of Embodiment 6 is used. The experiment is performed under the same conditions except for the decrease in the flow rate. The human serum is used as a sample carrier solution and a diluted solution for the standard sample, and the flow rate is maintained to be 1 μL/min. The standard sample in a concentration range of from 0 to 100 ng/mL is prepared. The sample (15 μL) is injected into the sensor chip for 900 seconds so as to induce the attachment reaction, and the phosphate buffer solution is injected for 420 seconds so as to induce the detachment reaction. As a cycle assay form, the assay of the standard sample is performed in the order of from a low concentration solution to a high concentration solution, and after that, the assay returns in the order of from the high concentration solution to the low concentration solution again. The operation of the assay system and the data editing are the same as described in Embodiment 4. After the completion of assay, as described above, the surface of the sensor is reproduced.

In FIG. 1, the concentration response of the sensor is in proportion to the concentration of the analyte (refer to FIG. 7 (A)). By comparing the pattern of the response with the result (refer to FIG. 5 (B)) obtained under the condition where the flow rate is faster by 10 times, it can be understood that the sensitivity of assay is about 0.1 ng/mL and the response time is maintained to be 640 seconds (with 95% of the final response as a reference). In addition, by comparing the graphs of the concentration response (refer to FIG. 7 (B)), it can be seen that there is no great difference within the tested flow rate range and there is nod difference between the concentration responses measured at the time of an increase in concentration of the analyte and at the time of a decrease in concentration. In particular, the transient response phenomenon (refer to the response at the concentration of the analyte of 10 ng/mL in FIG. 5) which occurs at the time of assay of a highly concentrated standard sample when the flow rate is relatively fast (10 μL/min) is greatly reduced if the flow rate is decreased (1 μL/min) (refer to the response at the concentration of the analyte of 10 ng/mL or more in FIG. 7).

### Embodiment 9

### Continuous Measurement According to Exponential Change in Concentration

In the aforementioned Embodiments, in order to measure the association and dissociation reactions of the reversible antibody, the reset mode where the sample carrier solution (excluding the analyte) is injected between the processes of the assay of the samples is used. On the contrary, in this Embodiment, in order to exemplify the recycling of the reversible antibody a sample continuous assay mode is used. The sensor chip manufactured in Embodiment 6 is used. The standard samples in a range of from 0.01 to 10,000 ng/mL are prepared by diluting the macroglobulin with human serum. The standard samples are sequentially injected at a flow rate of 1 μL/min into the sensor chip. The concentration response of the sensor is continuously obtained through repetition of two cycle changes where the concentration of the analyte is increased stepwise by 10 times every 900 seconds and decreased. With respect to the continuous mode of the sensor system, unlike the reset assay process set by the manufacturer of the sensor system, the injection of the sample is not performed through the inlet, but it is performed through the passage for supplying the sample carrier solution. The standard concentration of the next sample is adjusted by adding a predetermined concentrated or diluted solution of the analyte to the prior remaining sample solution so that there is no disconnection or air bubbles between the injections of the standard sample during the continuous supplying of the simple. Mixing is continually performed so that the concentration is uniform.

After the assay, the discharged sample is collected by a fractional collector. With respect to each fraction, the concentration of the analyte in the standard sample is checked by a sandwich enzyme-linked immunoassay using the plate of the micro well as a fixation maternal part. With respect to the assay method, the monoclonal antibody (1 μg/mL; 100 μL) of the irreversible antibody 3D1 having an irreversible reaction characteristic to the α2-macroglobulin diluted with 10 mM phosphate buffer solution (containing 140 mM NaCl; pH 7.4) is injected into each of the micro wells so as to perform the fixation. After washing, 10 mM phosphate buffer solution (casein-PBS; (200 μL) containing 0.5% casein is inserted so as to block the non-fixed remaining surface of the well. After washing again, 10 mM phosphate buffer solution (casein-twin-PBS: 70 μL) containing 0.5% casein and 0.1% twin is additionally injected to each of the fraction solutions (30 μL) collected according to the time by the fractional collector, so that the entire sample (100 μL) is inserted to react in the well where the antibody is fixed. After washing, 1 μg/mL 20E7-HRP polymer (100 μL) of the monoclonal antibody of the irreversible antibody 20E7 having an irreversible reaction characteristic to the α2-macroglobulin and the HRP is diluted by casein-twin-PBS and injected into the well so as to react. After washing again, an HRP substrate solution (refer to Embodiment 1) is added to each well, so that an enzyme reaction is performed. After 15 minutes, 2M sulfuric acid is added, and the reaction is allowed to stop. The color signal generated from each well is measured at the absorbance of 450 nm by using a micro plate reader (VERSAmax™, produced by Molecular Devices, USA).

The concentration of each of the standard samples, which is calculated and set for continuous measurement in advance, is collected after the continuous measurement. The actual concentration is checked through the aforementioned immunoassay. As a result, since the calculated values are different from the assay result within 10% or less, the calculated values are used for producing the graph. With respect to the response of the sensor according to an increase or decrease in concentration of the analyte in the standard sample which is injected into the sensor at a given micro flow rate (1 mL/min), the response commonly reaches the equilibrium state within 15 minutes or less, and high reproducibility is obtained in the two repetition cycle (refer to the result of test in a concentration range of from 0.01 to 100 ng/mL in FIG. 8 (A)). The standard curve (refer to FIG. 8 (B)) illustrating the concentration response of the sensor measured in continuous measurement mode is slightly different from the curve measured in the reset mode, which is determined to be caused from the difference between the operation methods of the sensor system. It can be understood from the result that the continuous measurement of the change in concentration the analyte can be actually performed and the continuous measurement can be applied to a clinical test.

### Embodiment 10

### Continuous Measurement According to Arithmetic Change in Concentration: Clinical Application As Childhood Renal Cancer Marker

Since the changing patterns of the concentration at the time of occurrence of disease or symptom may be different according to the type of the analyte, similarly to Embodiment 9, the concentration response of the sensor according to the arithmetic change in concentration, which is increased or decreased by twice or less, is measured in the continuous mode. In the experiment, the optimized conditions are used by taking into consideration diagnosis of the infantile renal cancer where the α2-macroglobulin selected as a model analyte can be used as a bio marker. In other words, the standard samples are manufactured by diluting the analyte with casein-PBS so that the consumption of serum sample can be minimized, and the concentration range thereof is determined to be in a range of from 1 to 20 ng/mL so that the assay performance can be maintained in the optimized state. The standard samples are injected into the sensor chip in a time interval of 1800 seconds, and the flow rate is adjusted to 1 μL/min.

As illustrated in FIG. 9, the response of the sensor according to the arithmetic continuous change the concentration exhibits a short response time and a good reproducibility of continuous measurement, similarly to the case of the exponential change in concentration. In addition, in view of a high sensitivity to a small change in concentration and a fast response, the reversible antibody-based bio sensor is expected to be widely used for measurement of analytes requiring very accurate assay. On the other hand, particularly, the clinically effective concentration range of the α2-macroglobulin is in a range of from 3 to 10 mg/mL. If the serum sample is directly used for the continuous measurement, 1.44 mL (with the injection rate of 1 μL/min as a reference) is consumed in a day. Since sample amount needs to be minimized, in this Embodiment, the sample is diluted so that concentration is lower by 10⁶ times. Therefore, in actual clinical test, serum can be consumed at a very small rate of about 1.44mL/day. Furthermore, under the assay conditions, the accuracy of assay, that is, the increase in the change width of signal according to the change in concentration of the analyte can be improved.

### INDUSTRIAL APPLICABILITY

As described above, according to the present invention, a change in concentration of an analyte can be measured in real time by continuously recycling a predetermined amount of a recognizing component having a reversible reaction characteristic. Therefore, by recycling an antibody which rapidly performs a reversible reaction according to a concentration of an analyte, configurations and manufacturing methods can be efficiently simplified in comparison with existing disposable diagnosis chip. In addition, since disease or symptoms can be monitored in real time, it is possible to continuously monitor chronic disease or high risk group patients. In addition, the present invention can be applied to an artificial organ control device, a continuous detecting system for a biological terror agent a continuous detecting system for a zoonotic infection pathogen, a continuous detecting system for an environment contaminant, a continuous detecting system for a biological process, a continuous detecting system for a food producing process, or the like.

1. A real-time continuous detection device for detecting an analyte in a sample comprising:
a sample inflow channel;
a sample assay site; and
a sample outflow channel,
wherein the sample assay site includes a reversible capturing recognizing component and a sensor which detects a signal generated from a binding body of the analyte and the reversible capturing recognizing component.

2. The real-time continuous detection device according to claim 1, wherein the reversible capturing recognizing component has a reversible reaction characteristic so that an association rate constant kₐ is in a range of from 1×10⁵ Lmol⁻¹ sec⁻¹ to 1×10⁸ Lmol⁻¹ sec⁻¹ and a dissociation rate constant k_{d} is in a range of from 1×10⁻³ sec⁻¹ to 1×10⁻¹ sec⁻¹ and a high affinity so that an equilibrium association constant K_{A}=kₐ/k_{d} is 1×10⁸ L/mol or more at the time of reacting with the analyte in the sample.

3. The real-time continuous detection device according to claim 1, wherein the reversible capturing recognizing component is an antibody, a receptor, a nucleic acid, an enzyme, an aptamer, a peptide, or a molecular printing artificial membrane which can specifically bind to the analyte in the sample such as living organism metabolites, a protein, a hormone, a nucleic acid, a cell, a food test material, an environment contaminant, or national-defense chemical, biological and radiological test materials.

4. The real-time continuous detection device according to claim 1, wherein the sensor is a label-free sensor which directly detects the signal generated from the binding body of the analyte and the capturing recognizing component or a label sensor which performs detection through a label material generating the signal in proportion to a density of the binding body of the analyte and the capturing recognizing component.

5. The real-time continuous detection device according to claim 4, wherein the label-free sensor is a surface plasmon resonance sensor, a cantilever sensor, an optical waveguide sensor, an optical interference sensor, or a nanosensor.

6. The real-time continuous detection device according to claim 4, wherein the label sensor is a fluorescence sensor, a luminescence sensor, a color sensor, an electro-chemical sensor, or a magnetic field detecting sensor which uses a fluorescent material, a luminescent material, an enzyme, a metal particle, a plastic particle, a magnetic particle, or a nanoparticle as a label material.

7. The real-time continuous detection device according to claim 1, wherein the sample assay site is partitioned by a semi-permeable membrane which can selectively permeate only the analyte in the sample so that a recognizing reaction cell is formed to the side of the surface of the sensor where the capturing recognizing component is fixed.

8. The real-time continuous detection device according to claim 7, wherein in the case of using the label sensor, a detecting recognizing component which is bound with the label material, which cannot permeate through the semi-permeable membrane in size, is confined in the recognizing reaction cell so as to be recycled.

9. The real-time continuous detection device according to claim 8, wherein the detecting recognizing component and the capturing recognizing component in the recognizing reaction cell have reversible reaction characteristics so as to be continuously recycled.

10. A real-time continuous detection method for detecting an analyte using the real-time continuous detection device according to claim 1, comprising steps of:
(a) injecting the sample containing the analyte through the sample inflow channel into the sample assay site;
(b) binding the analyte with the reversible capturing recognizing component in the sample assay site;
(c) detecting the signal generated from the binding body of the analyte and the capturing recognizing component by using the sensor;
(d) detaching the analyte from the capturing recognizing component and discharging the analyte through the sample outflow channel by a continuous inflow of the sample or an inflow of a washing solution; and
(e) repeating the steps (b) to (d) by recycling the detached capturing recognizing component, so that a change in concentration of the analyte in the sample is measured in real time.

11. The real-time continuous detection method according to claim 10, wherein in the step (c), the signal generated from the binding body of the analyte and the capturing recognizing component is directly detected by using a label-free sensor, or the signal is measured through a label material generating the signal in proportion to a density of the binding body of the analyte and the capturing recognizing component by using a label sensor.

12. The real-time continuous detection method according to claim 11, wherein in the case of using the label-free sensor, the analyte included in the sample is continuously flown through the sample inflow channel into the sample assay site to react with the capturing recognizing component.

13. The real-time continuous detection method according to claim 11, wherein in the case of using the label sensor, after the analyte in the sample reacts with the detecting recognizing component bound with the label material in advance, the analyte is continuously flown through the sample inflow channel into the sample assay site to react with the capturing recognizing component (continuous flow exposure type), or after the analyte is continuously flown through the sample inflow channel into the sample assay site, the analyte reacts with the capturing recognizing component and the detecting recognizing component bound with the label material in the recognizing reaction cell (recognizing reaction cell type).

14. The real-time continuous detection method according to claim 13, wherein in the case of the continuous flow exposure type, the detecting recognizing component that reacts with the analyte in advance has an irreversible reaction characteristic with high binding stability, and in the case of the recognizing reaction cell type, the detecting recognizing component has a reversible reaction characteristic so that the capturing recognizing component and the detecting recognizing component can be continuously recycled.

15. The real-time continuous detection method according to claim 13, wherein in the case of using the recognizing reaction cell type label sensor, the recognizing reaction can be performed in liquid state without fixation of the capturing recognizing component on the surface of the sensor by using a principle that a fluorescence signal is generated due to interference to energy transfer between neighboring fluorescence material (label material) and fluorescence energy receptor by reaction of the capturing recognizing component and the analyte, or by using an enzyme, of which the activity is known to be suppressed by the binding of the capturing recognizing component and the analyte fixed on the enzyme molecule (label material), as the label material.

16. A method of selecting a reversible capturing recognizing component used for the real-time continuous detection device according to claim 1, comprising steps of:
(a) preparing the capturing recognizing component;
(b) binding the capturing recognizing component with the analyte fixed on the surface of the sensor;
(c) detecting the signal generated from the binding body of the capturing recognizing component and the analyte by using the sensor;
(d) detaching the analyte from the capturing recognizing component by an inflow of a washing solution;
(e) detecting a signal generated from the binding body of the capturing recognizing component and the analyte remained after the detaching by the sensor; and
(f) selecting the capturing recognizing component of which the signal detected in the step (e) is lower than the signal detected in the step (c).

17. The method according to claim 16, wherein the sensor is a label-free sensor selected from a surface plasmon resonance sensor, a cantilever sensor, an optical waveguide sensor, an optical interference sensor, and a nanosensor.

18. The method according to claim 16, wherein the capturing recognizing component has a reversible reaction characteristic so that an association rate constant kₐ is in a range of from 1×10⁵ Lmol⁻¹ sec⁻¹ to 1×10⁸ Lmol⁻¹ sec⁻¹ and a dissociation rate constant k_{d} is in a range of from 1×10⁻³ sec⁻¹ to 1×10⁻¹ sec⁻¹ and a high affinity so that an equilibrium association constant K_{A}=kₐ/k_{d} is 1×10⁸ L/mol or more at the time of reacting with the analyte in the sample.

19. The method according to claim 16, wherein, in the step (a), the capturing recognizing component is diluted with a carrier solution and continuously injected, and in the step (f), the capturing recognizing component generating the signal pattern, where the signal is increased and then decreased as the time elapses, is selected.

20. The method according to claim 16, wherein, in the step (a), an alternative injection of the capturing recognizing component and a washing solution is repeated, and in the step (f), the capturing recognizing component generating the signal pattern, where the signal is increased and then returns to an initial base line repeatedly as the time elapses, is selected.
